SEQUENCE LISTING (1 sequence)

SEQ ID NO: 1 - RNA, 1517 nt, Parabacteroides goldsteinii
  source 1..1517: mol_type = rRNA
gtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacctccttt

(19) United States
(12) Patent Application Publication
(10) Pub. No.: US 2023/0364160 A1
(43) Pub. Date: Nov. 16, 2023

(54) METHOD FOR TREATING PARKINSON’S DISEASE BY USING PARABACTEROIDES GOLDSTEINII

(71) Applicants: Multistars Biotechnology Company Limited, Taoyuan City (TW); CHANG GUNG UNIVERSITY, Taoyuan City (TW); NATIONAL TAIWAN UNIVERSITY HOSPITAL, Taipei City (TW)

(72) Inventors: Chin-Hsien Lin, Taipei City (TW); Hsin-Chih Lai, Taoyuan City (TW); Chia-Chen Lu, Taoyuan City (TW); Tzu-Lung Lin, Taoyuan City (TW); Po-I Wu, Taoyuan City (TW)

(21) Appl. No.: 18/196,574

(22) Filed: May 12, 2023

Related U.S. Application Data
(60) Provisional application No. 63/341,437, filed on May 13, 2022.

(51) Int. Cl. A61K 35/74; C12N 1/20; A61P 25/28

(52) U.S. Cl. CPC A61K 35/74; C12N 1/20; A61P 25/28

(57) ABSTRACT

The present disclosure provides a method for treating Parkinson's disease by using Parabacteroides goldsteinii. The Parabacteroides goldsteinii of the present disclosure achieves the effect of treating Parkinson's disease through various efficacy experiments.

### CROSS-REFERENCE TO RELATED APPLICATION

This application claims priority of Provisional Application No. 63/341,437, filed on May 13, 2022, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 112F0101-IE_Sequence_listing. The XML file is 4000 bytes; was created on May 12, 2023.

### BACKGROUND OF THE INVENTION

### 1. Field of the Invention

The present invention relates to a method for treating Parkinson's disease by using Parabacteroides goldsteinii.

### 2. The Prior Art

Parkinson's disease belongs to a movement disorder, and is prone to occur in people over the age of 50 or 60. The symptoms of the manifestation are mainly resting tremors of limbs, muscle stiffness, slow movement, and unstable posture leading to balance disorders. Early symptoms often include regular shaking of one hand, slow movements, and muscle stiffness gradually spread throughout the body. In the final stage, it is almost impossible to move around in a wheelchair. Parkinson's disease is a dopamine deficiency due to the death of neurons that release dopamine as a neurotransmitter in the substantia nigra pars compacta located in the midbrain. The striatum in the basal ganglia, which relies on the substantia nigra cells to provide dopamine as a neurotransmitter, is affected, unable to regulate the messages of the cerebral cortex, thalamus and extrapyramidal system, thus causing dysfunction in the coordination and operation of the muscles. Like Alzheimer's, Parkinson's disease has no cure. At present, patients with Parkinson's disease are relieved clinically by supplementing L-dopamine, but the use of L-dopamine often causes side effects. If the symptoms are more serious and cannot be controlled by drugs, you can also consider implanting electrodes in the brain for deep brain stimulation, but this is highly invasive and uncertain for brain surgery. Because the neurons continue to die, no matter whether drugs or surgery can only relieve some symptoms, but have no therapeutic function.

At present, clinical Parkinson's disease drug treatment has limited effect and has serious side effects, and many patients cannot continue to treat. More importantly, the drug only alleviates the symptoms, but fails to fundamentally solve the problem of neurodegeneration and death, so how to develop a new drug that can really treat Parkinson's disease is an important issue that the present invention intends to solve here.

In order to solve the above-mentioned problems, those skilled in the art urgently need to develop a novel pharmaceutical composition for treating Parkinson's disease for the benefit of a large group of people in need thereof.

### SUMMARY OF THE INVENTION

A primary objective of the present invention is to provide a method for treating Parkinson's disease, comprising administering to a subject in need thereof a pharmaceutical composition comprising an effective amount of Parabacteroides goldsteinii.

According to an embodiment of the present invention, the Parabacteroides goldsteinii is a live bacterium.

According to an embodiment of the present invention, the effective amount of Parabacteroides goldsteinii is at least 2×10⁸ CFUs per day for the subject.

According to an embodiment of the present invention, the Parabacteroides goldsteinii improves locomotor function of the subject in need of treatment for Parkinson's disease.

According to an embodiment of the present invention, the Parabacteroides goldsteinii mitigates dopaminergic neuronal degeneration and microglial activation of the subject in need of treatment for Parkinson's disease.

According to an embodiment of the present invention, the Parabacteroides goldsteinii alleviates neuroinflammation of the subject in need of treatment for Parkinson's disease.

According to an embodiment of the present invention, the Parabacteroides goldsteinii activates mitochondrial function of the subject in need of treatment for Parkinson's disease.

According to an embodiment of the present invention, the pharmaceutical composition is in a dosage form for oral administration.

According to an embodiment of the present invention, the pharmaceutical composition is in a dosage form for parenteral administration.

According to an embodiment of the present invention, the pharmaceutical composition further comprises a pharmaceutically acceptable excipient, carrier, adjuvant and/or food additive.

According to an embodiment of the present invention, the pharmaceutical composition is taken together with general food, the pharmaceutical composition further comprises bacteria other than the Parabacteroides goldsteinii, and the pharmaceutical composition further comprises an ingredient selected from the group consisting of proteins, monosaccharides, disaccharides, oligosaccharides, polysaccharides, carbohydrates, amino acids, lipids, vitamins, and any combination thereof.

According to an embodiment of the present invention, the Parabacteroides goldsteinii is a Parabacteroides goldsteinii strain, and the Parabacteroides goldsteinii strain comprises a 16s rRNA gene sequence with at least 95%, 96%, 97%, 98%, 99%, 99.5%, or 99.9% sequence identity to the 16s rRNA gene sequence of Parabacteroides goldsteinii strain MTS01.

In summary, the Parabacteroides goldsteinii of the present invention achieves the effect on treating Parkinson's disease through improving locomotor function of the subject in need of treatment for Parkinson's disease, mitigating dopaminergic neuronal degeneration and microglial activation of the subject in need of treatment for Parkinson's disease, alleviating neuroinflammation of the subject in need of treatment for Parkinson's disease, and activating mitochondrial function of the subject in need of treatment for Parkinson's disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included here to further demonstrate some aspects of the present invention, which can be better understood by reference to one or more of these drawings, in combination with the detailed description of the embodiments presented herein.

FIGS. 1A, 1B, and 1C show that Parabacteroides goldsteinii strain MTS01 (PG) implantation improves motor dysfunction. (1A) Total moving distance of open field assay, (1B) Mean moving velocity in the open field, (1C) There was no significant body weight difference between groups. Animals tested serially from 5 month to 10 months and the Parabacteroides goldsteinii strain MTS01 was inoculated at the age of 5 months. N=10-21 in each group, error bars represent mean from 3 trials per animal Data representative of 2 repeats per experiment. SPF means specific pathogen free, GF means germ-free, NTG means non-transgenic, and PG means Parabacteroides goldsteinii strain MTS01.

FIGS. 2A and 2B show that Parabacteroides goldsteinii strain MTS01 (PG) implantation restores locomotor defects in Parkinson's disease (PD) mice. Germ free status slightly improves motor function in LRRK2 G2019S mice compared to SPF status, and furthermore, inoculation of Parabacteroides goldsteinii strain MTS01 significantly rescured the motor dysfunction of LRRK2 G2019S mice in both moving distance (2A) and moving velocity (2B). N=10-21 in each group, error bars represent mean from 3 trials per animal Data representative of 2 repeats per experiment. SPF, specific pathogen free; GF, germ free; PG, inoculation of Parabacteroides goldsteinii strain MTS01. *P≤0.05; **P≤0.01; ***P≤0.001.

FIG. 3 shows that Parabacteroides goldsteinii strain MTS01 (PG) implantation improves tyrosine hydroxylase (TH) (+) cell loss in the substantia nigra. The number of nigral TH (+) cells was markedly reduced in LRRK2 G2019S mice (B) compared to NTG wild type animals under the specific pathogen free (SPF) condition. Under germ-free (GF) condition, LRRK2 G2019S mice (D) display appreciably more dopaminergic neuronal loss in these regions than NTG wild type animals (C). Remarkably, GF-LRRK2 G2019S mice with inoculation of Parabacteroides goldsteinii strain MTS01 (F) exhibit a significantly increased number of TH(+) cells in this region compared to SPF-LRRK2 G2019S mice or GF-LRRK2 G2019S mice (E). The number of TH(+) cells in substantia nigra of different groups is shown (G). *P≤0.05; **P≤0.01. DAPI means 4′,6-diamidino-2-phenylindole, which is a blue fluorescent dye, which is a fluorescent dye that can strongly bind to DNA. Iba1 is a calcium-binding protein expressed exclusively in macrophages/microglia. LRRK2 means Leucine-rich repeat kinase 2, GF means germ-free, PG means inoculation of Parabacteroides goldsteinii strain MTS01.

FIG. 4 shows that Parabacteroides goldsteinii strain MTS01 (PG) implantation improves Iba1-positive cell activation in LRRK2 G2019S mice. Under germ-free condition, microglia of GF-LRRK2 G2019S mice display microglia activation compared to GF-NTG wild type mice (A, B). After inoculation with Parabacteroides goldsteinii strain MTS01, the activated microglia in the nigral region of LRRK2 G2019S mice returned to be a non-active state (C, D). (E, F) The morphology analysis using Sholl analysis of microglia in individual group of mice. DAPI means 4′,6-diamidino-2-phenylindole, which is a blue fluorescent dye, which is a fluorescent dye that can strongly bind to DNA. Iba1 is a calcium-binding protein expressed exclusively in macrophages/microglia. LRRK2 means Leucine-rich repeat kinase 2, GF means germ-free, PG means inoculation of Parabacteroides goldsteinii strain MTS01.

FIGS. 5A-5D show that Parabacteroides goldsteinii strain MTS01 (PG) implantation causes gene cluster changes in brain tissue RNA sequences. (5A) changes of gene clusters in LRRK2 G2019S mice with and without PG inoculation. (5B,5C,5D). Parabacteroides goldsteinii strain MTS01 diminished brain inflammation via toll-like receptor (TLR) and nuclear factor kappa B (NF-κB) pathways. N=4 in each group. LRRK2 means Leucine-rich repeat kinase 2.

FIGS. 6A-6D show that Parabacteroides goldsteinii strain MTS01 (PG) implantation did not affect gut gene clusters related to inflammatory responses (A), TLR signaling pathways (B) or NF-κB pathways (C) of LRRK2 G2019S mice. Notably, Parabacteroides goldsteinii strain MTS01 activates mitochondrial function in gut tissues of LRRK2 G2019S mice (D). N=4 in each group. LRRK2 means Leucine-rich repeat kinase 2. The four colors represent the four gene-enriched features in the upper right corner of each figure.

FIG. 7 shows the mitochondrial respiratory function of colon tissues of germ-free LRRK2 G2019S mice and those mono-colonized with Parabacteroides goldsteinii strain MTS01. The rate of oxygen consumption (OCR) of isolated mitochondria was determined with the Seahorse XF24 analyzer. The data was analyzed by unpaired t test (n=5 in each group).

FIG. 8 shows the circular genome map of the whole-genome sequence analysis of the Parabacteroides goldsteinii strain MTS01.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

As used herein, the term “effective dose” refers to the amount of Parabacteroides goldsteinii required for treating Parkinson's disease. The effective dose may be different depending on the biological species or individual differences to be treated, but the effective dose can be determined experimentally by, for example, dose escalation.

According to the present invention, the operating procedures and parameter conditions related to bacterial culture fall within the scope of the professional literacy and routine techniques of those skilled in the art.

As used herein, the term “bacterial component” refers to the derivative substances directly or indirectly related to the bacteria when the bacteria are cultivated, including but not limited to the metabolites of the bacteria, the structure of the bacteria, active and inactive components related to the bacteria, etc.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

According to the present invention, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

### Example 1

Parabacteroides goldsteinii Strain MTS01 Cultivation and Evaluation of Effect of Parabacteroides goldsteinii Strain MTS01 on Improving Locomotor Function of Subject in Need of Treatment for Parkinson's Disease

The cultivation procedure of Parabacteroides goldsteinii strain MTS01 is as follows. The Parabacteroides goldsteinii strain MTS01 was deposited in the Deutsche Sammlung von Mikroorganismen and Zellkulturen (DSMZ) on Oct. 29, 2018, under accession number DSM 32939. The Parabacteroides goldsteinii strain MTS01 in the present application is a strain that is easily obtained by those ordinarily skilled in the art, and has been disclosed in U.S. application Ser. No. 16/541,308 (corresponding to U.S. Pat. No. 11,166,988). Parabacteroides goldsteinii strain MTS01 was cultivated on anaerobic blood agar (Creative, New Taipei city, Taiwan) and liquid thioglycollate medium (BD, Franklin Lakes, USA) at 37° C. in a Whitley DG250 anaerobic chamber (Don Whitley, Bingley, UK) with mixed anaerobic gas (5% carbon dioxide, 5% hydrogen, 90% nitrogen). Anaerobic condition was confirmed using an anaerobic indicator (Oxoid, Hampshire, UK). The Parabacteroides goldsteinii strain MTS01 will be publicly available upon the granting of the present application.

The experimental process for establishing germ-free PD mouse model having the LRRK2 G2019S mutation is as follows. We established a germ-free PD genetic rodent model and then inoculate the Parabacteroides goldsteinii strain MTS01 to examine its potential beneficial effects on neurodegeneration and locomotor defects in the process of PD. The PD genetic rodent model is the transgenic LRRK2 G2019S mice (FVB/N-Tg [LRRK2*G2019S]1Cjli/J, JAX009609, The Jackson Laboratory, Bar Harbor, ME USA), which we had characterized the phenotypes and neuropathology thoroughly (see Liang F, Chen C Y, Li Y P, Ke Y C, Ho E P, Jeng C F, Lin C H, Chen S K: Early Dysbiosis and Dampened Gut Microbe Oscillation Precede Motor Dysfunction and Neuropathology in Animal Models of Parkinson's Disease. J Parkinsons Dis 2022, 12(8):2423-2440). The LRRK2 heterozygous mice were then intercrossed to obtain non-transgenic (NTG) littermate control animals. Hemizygous mice develop adult-onset age-dependent locomotor dysfunction between 10-11 months of age leading to death within 6 months of onset of motor symptoms. Affected mice exhibit neuronal age-dependent dopaminergic neuronal apoptosis and locomotor, mimicking human PD. The colonic and brain expression of α-synuclein of LRRK2 G2019S mice were increased in the chronic intestinal inflammation status compared to non-colitis status or NTG controls (see Lin C H, Lin H Y, Ho E P, Ke Y C, Cheng M F, Shiue C Y, Wu C H, Liao P H, Hsu A Y, Chu L A et al: Mild Chronic Colitis Triggers Parkinsonism in LRRK2 Mutant Mice Through Activating TNF-alpha Pathway. Mov Disord 2022, 37(4):745-757).

The transgenic LRRK2 G2019S mice were housed in sterilized isolators for the germ-free process in the National Laboratory Animal Center. Germ-free LRRK2 G2019S mice were maintained in the gnotobiotic facility of the National Laboratory Animal Center and will be routinely tested for microbes and parasites to ensure germ-free conditions.

The experimental process for inoculation of Parabacteroides goldsteinii strain MTS01 in germ-free PD mouse model is as follows. At the age of 5 months old, we inoculated the Parabacteroides goldsteinii strain MTS01 (2×10⁸ CFU per mouse) into the germ-free heterozygous LRRK2 G2019S mice. The control animals were littermate NTG mice and those without Parabacteroides goldsteinii strain MTS01 implantation. Serial motor function evaluation every month using rotarod, open filed test and catwalk analysis were done until the age of 10 months old. We monthly examined the locomotor function, including open filed test, rotaroad, and catwalk analysis. A total of 20-30 germ-free mice (5-7 mice per group) were used in the present invention.

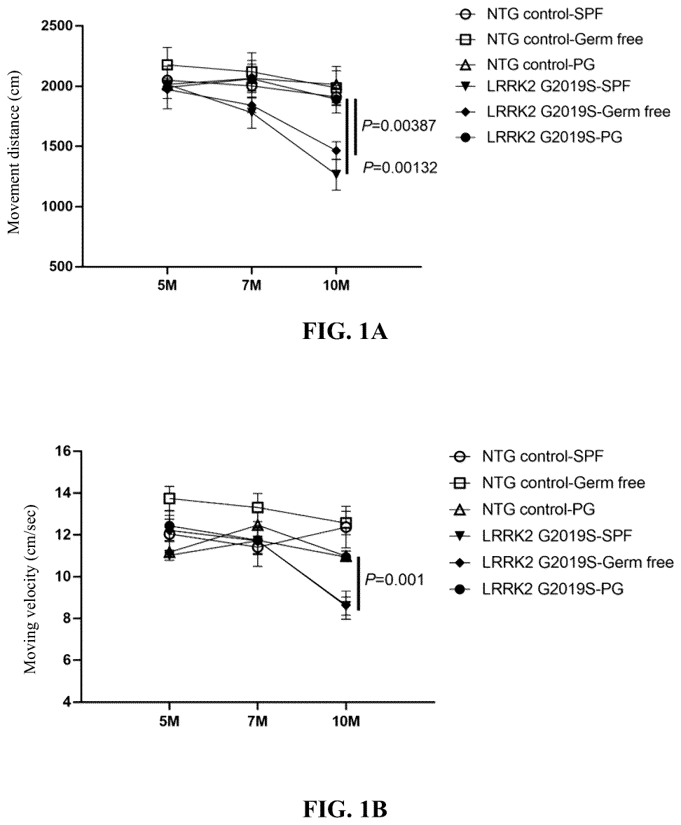

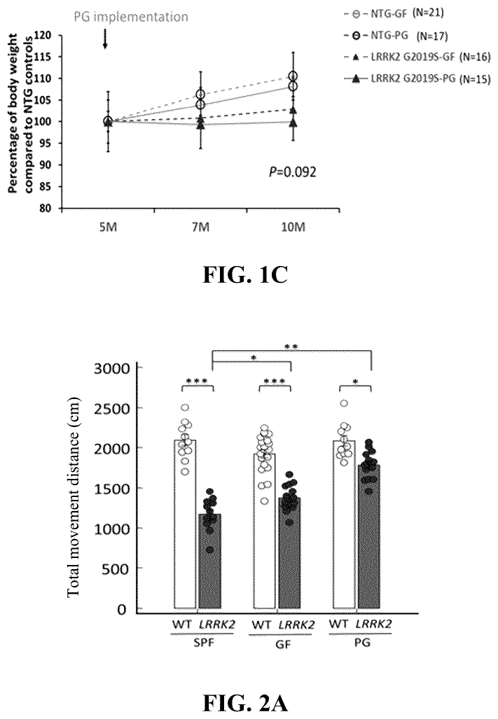

The experimental process for motor function assay is as follows. We compared the locomotor function between each group monthly to see whether the inoculation of Parabacteroides goldsteinii strain MTS01 improves the motor defects of LRRK2 G2019S mice when they aged. The locomotor function assay included rotaroad, open filed assay and cat walking analysis. The body weight, stool passage and gastrointestinal motility were recorded monthly. The result is shown in FIGS. 1A-1C.

FIGS. 1A and 1B show that Parabacteroides goldsteinii strain MTS01 (PG) implantation improves motor dysfunction. (1A) Total moving distance of open field assay, (1B) Mean moving velocity in the open field, (1C) There was no significant body weight difference between groups. Animals tested serially from 5 month to 10 months and the Parabacteroides goldsteinii strain MTS01 was inoculated at the age of 5 months. N=10-21 in each group, error bars represent mean from 3 trials per animal Data representative of 2 repeats per experiment. SPF means specific pathogen free, GF means germ-free, NTG means non-transgenic, and PG means Parabacteroides goldsteinii strain MTS01.

The LRRK2 G2019S mouse model displays progressive age-dependent deficits in motor function similar to human Parkinson disease (PD) in terms of moving distance and moving velocity compared to non-transgenic controls (see FIGS. 1A and 1B). To assess a contribution of gut bacteria, we re-derived LRRK2 G2019S mice under germ-free conditions (GF) and compared performance in these tests to those with inoculation with Parabacteroides goldsteinii strain MTS01 (PG). Of note, GF-LRRK2 G2019S animals exhibit reduced deficits in the moving distance and total moving time to cross the open filed and catwalk platform (see FIGS. 1A and 1B). Remarkably, GF-LRRK2 G2019S mice with Parabacteroides goldsteinii strain MTS01 (PG) exhibit a significantly improved moving time and moving distance compared to GF-LRRK2 G2019S mice or NTG control animals (see FIGS. 1A and 1B). There was no significant body weight difference between groups (see FIG. 1C).

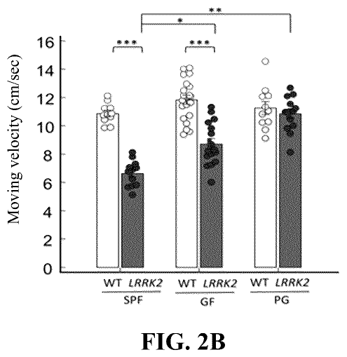

FIGS. 2A and 2B show that Parabacteroides goldsteinii strain MTS01 (PG) implantation restores locomotor defects in Parkinson's disease (PD) mice. Germ free status slightly improves motor function in LRRK2 G2019S mice compared to SPF status, and furthermore, inoculation of Parabacteroides goldsteinii strain MTS01 significantly rescured the motor dysfunction of LRRK2 G2019S mice in both moving distance (2A) and moving velocity (2B). N=10-21 in each group, error bars represent mean from 3 trials per animal Data representative of 2 repeats per experiment. SPF, specific pathogen free; GF, germ free; PG, inoculation of Parabacteroides goldsteinii strain MTS01. *P≤0.05; **P≤0.01; ***P≤0.001.

Specifically, 5 months after Parabacteroides goldsteinii strain MTS01 (PG) treatment at the age of 10 months old, germ free LRRK2 G2019S mice after inoculation of Parabacteroides goldsteinii strain MTS01 (PG) exhibit a remarkably improved moving distance and moving velocity compared to SPF-LRRK2 G2019S mice or NTG control animals (see FIGS. 2A and 2B).

These results suggest that inoculation of Parabacteroides goldsteinii strain MTS01 mitigated age-dependent locomotor defects of PD.

### Example 2

Inoculation of Parabacteroides goldsteinii Strain MTS01 Mitigated Dopaminergic Neuronal Degeneration and Microglial Activation of Subject in Need of Treatment for Parkinson's Disease

Motor deficits in PD coincide with the dopaminergic neuronal degeneration and microglia activation in the transgenic LRRK2 G2019S mice (see Lin C H, Lin H Y, Ho E P, Ke Y C, Cheng M F, Shiue C Y, Wu C H, Liao P H, Hsu A Y, Chu L A et al: Mild Chronic Colitis Triggers Parkinsonism in LRRK2 Mutant Mice Through Activating TNF-alpha Pathway. Mov Disord 2022, 37(4):745-757).

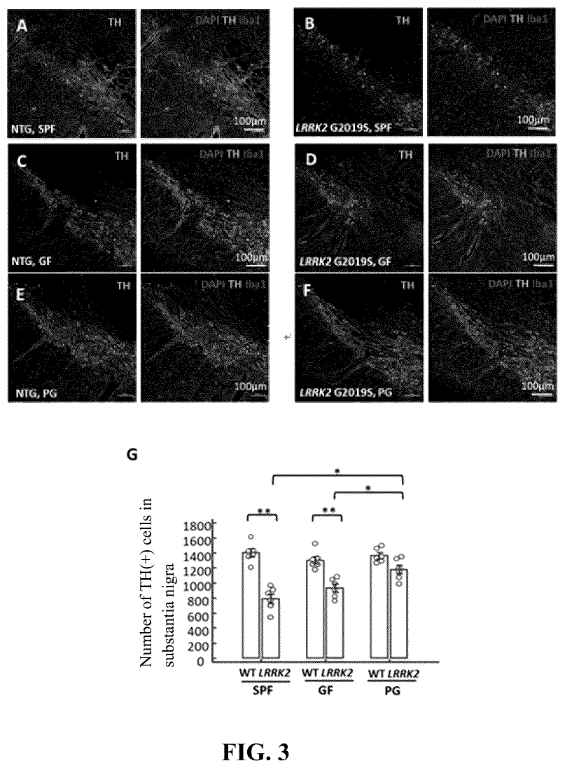

FIG. 3 shows that Parabacteroides goldsteinii strain MTS01 (PG) implantation improves tyrosine hydroxylase (TH) (+) cell loss in the substantia nigra. The number of nigral TH (+) cells was markedly reduced in LRRK2 G2019S mice (B) compared to NTG wild type animals under the specific pathogen free (SPF) condition. Under germ-free (GF) condition, LRRK2 G2019S mice (D) display appreciably more dopaminergic neuronal loss in these regions than NTG wild type animals (C). Remarkably, GF-LRRK2 G2019S mice with inoculation of Parabacteroides goldsteinii strain MTS01 (F) exhibit a significantly increased number of TH(+) cells in this region compared to SPF-LRRK2 G2019S mice or GF-LRRK2 G2019S mice (E). The number of TH(+) cells in substantia nigra of different groups is shown (G). *P≤0.05; **P≤0.01. DAPI means 4′,6-diamidino-2-phenylindole, which is a blue fluorescent dye, which is a fluorescent dye that can strongly bind to DNA. Iba1 is a calcium-binding protein expressed exclusively in macrophages/microglia. LRRK2 means Leucine-rich repeat kinase 2, GF means germ-free, PG means inoculation of Parabacteroides goldsteinii strain MTS01.

Under specific pathogen free (SPF) conditions, we observed the dopaminergic neuron labelled as anti-TH (+) in the substantia nigra was markedly reduced in LRRK2 G2019S compared to NTG wild type animals (see FIGS. 3A, B, statistics in G). Surprisingly, GF-LRRK2 G2019S mice display appreciably less dopaminergic neuronal loss in these regions (FIGS. 3C, D, statistics in G). Remarkably, GF-LRRK2 G2019S mice with inoculation of Parabacteroides goldsteinii strain MTS01 (PG) exhibit a significantly increased number of TH (+) cells in this region compared to SPF-LRRK2 G2019S mice or GF-LRRK2 G2019S mice and nearly reach to the level of or NTG control animals (FIGS. 3E, F, statistics in G).

Neuroinflammation is also a pathological feature of PD and the LRRK2 G2019S mice is prone to inflammatory status (see Lin C H, Lin H Y, Ho E P, Ke Y C, Cheng M F, Shiue C Y, Wu C H, Liao P H, Hsu A Y, Chu L A et al: Mild Chronic Colitis Triggers Parkinsonism in LRRK2 Mutant Mice Through Activating TNF-alpha Pathway. Mov Disord 2022, 37(4):745-757). Microglia is a brain-resident immune cell, and microglia undergo significant morphological changes upon activation, transitioning from thin cell bodies with numerous branched extensions to round, amoeboid cells with fewer processes.

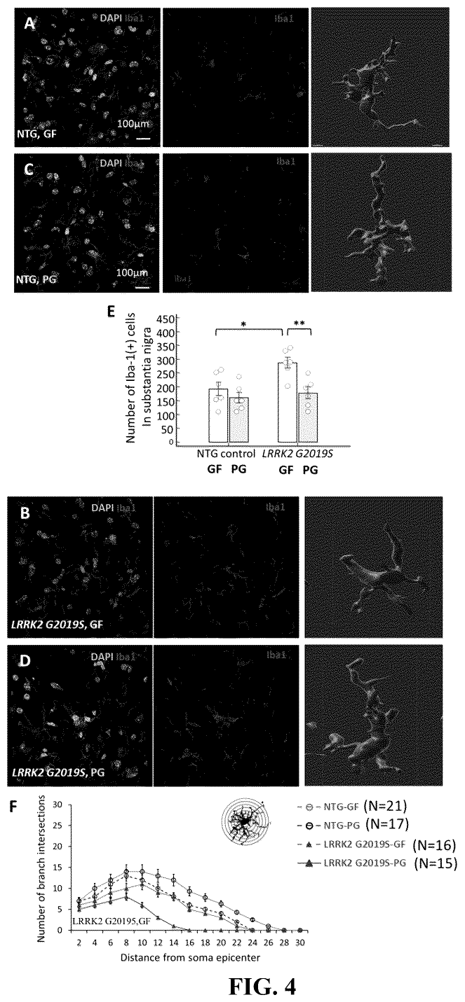

FIG. 4 shows that Parabacteroides goldsteinii strain MTS01 (PG) implantation improves Iba1-positive cell activation in LRRK2 G2019S mice. Under germ-free condition, microglia of GF-LRRK2 G2019S mice display microglia activation compared to GF-NTG wild type mice (A, B). After inoculation with Parabacteroides goldsteinii strain MTS01, the activated microglia in the nigral region of LRRK2 G2019S mice returned to be a non-active state (C, D). (E, F) The morphology analysis using Sholl analysis of microglia in individual group of mice. DAPI means 4′,6-diamidino-2-phenylindole, which is a blue fluorescent dye, which is a fluorescent dye that can strongly bind to DNA. Iba1 is a calcium-binding protein expressed exclusively in macrophages/microglia. LRRK2 means Leucine-rich repeat kinase 2, GF means germ-free, PG means inoculation of Parabacteroides goldsteinii strain MTS01.

Under germ-free condition, within the substantia, microglia of GF-LRRK2 G2019S mice display significant increases in cell body diameter, along with fewer processes of shorter total length, compared to GF-NTG wild type mice (FIGS. 4A, B). Notably, after inoculation with Parabacteroides goldsteinii strain MTS01, the activated microglia in the nigral region of LRRK2 G2019S mice returned to be a non-active state, which presents with decreases in cell body diameter, along with more processes of longer total length, compared to NTG wild type mice with Parabacteroides goldsteinii strain MTS01 or GF-LRRK2 G2019S mice (FIGS. 4C, D and statistics in F). Consistently, the number of Iba-1 (+) microglia was increased in GF-LRRK2 G2019S mice compared to NTG wild type mice and then the number reduced after treatment (FIG. 4E).

### Example 3

Gut Inoculation of Parabacteroides goldsteinii Strain MTS01 Alleviates Neuroinflammation of Subject in Need of Treatment for Parkinson's Disease

After inoculation with Parabacteroides goldsteinii strain MTS01, the activated microglia in the nigral region of LRRK2 G2019S mice returned to be a non-active state (FIG. 4). The increased number of Iba-1 (+) microglia in GF-LRRK2 G2019S mice reduced after inoculation with Parabacteroides goldsteinii strain MTS01, suggesting this microbiota alleviates neuroinflammation.

The experimental process of the transcriptome analysis of gut and brain tissues after Parabacteroides goldsteinii strain MTS01 treatment is as follows. To investigate the possible mechanisms of Parabacteroides goldsteinii strain MTS01 on gut and dopaminergic neuronal function, we performed bulk RNA-seq on mouse gut and brain tissues after Parabacteroides goldsteinii strain MTS01 treatment. Total RNA was extracted from the harvested tissues and the extracted RNA quality was assessed to determine whether the samples are suitable for RNA-seq. Libraries were generated by KAPA RNA HyperPrep Kit with RiboErase according to the manufacturer's instructions and were sequenced by the Illumina NovaSeq. Raw reads were quality checked by FastQC. Adaptor sequences were trimmed, and low-quality reads were filtered out using cutadapt. The qualified reads were mapped to mouse reference genome GRCm38 by STAR algorithm, and gene expression were quantified based on the GENCODE gene model. Differential expression analysis was performed according to the limma-voom pipeline. Over-representation analysis and gene-set enrichment analysis (GSEA) on gene-sets from MSigDB were performed using the R package clusterProfiler.

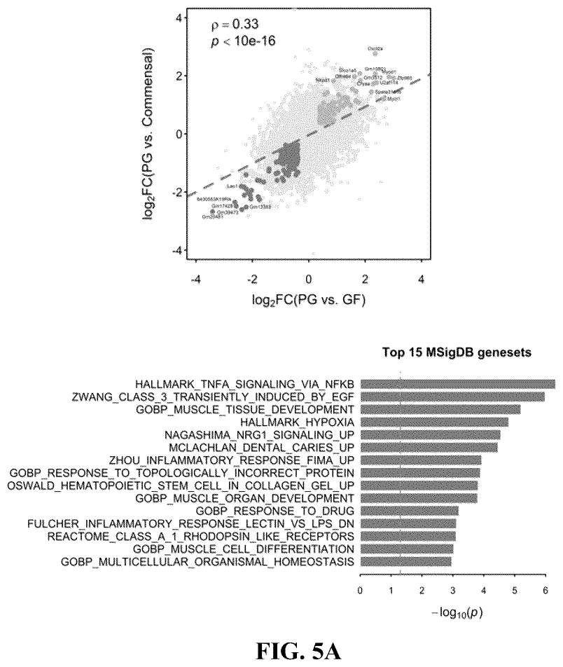

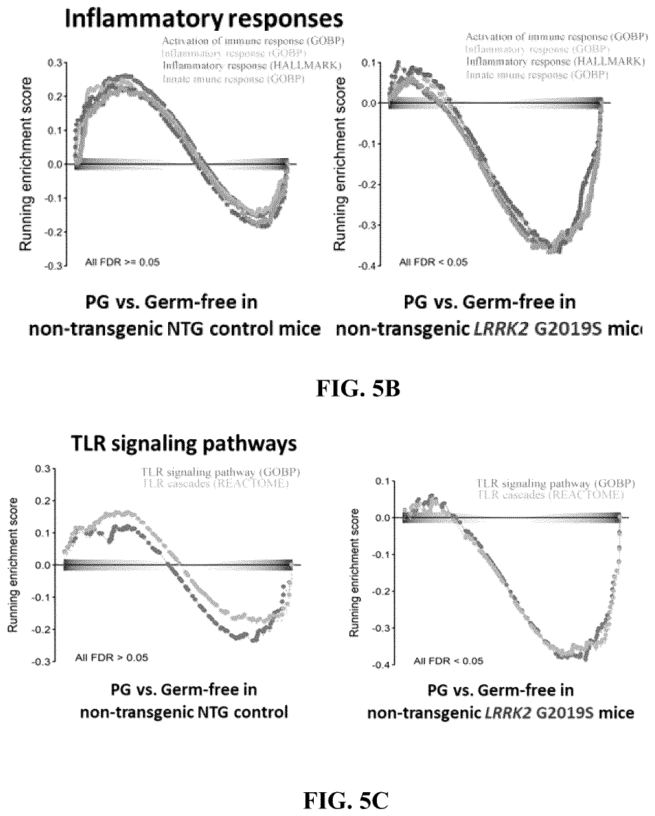

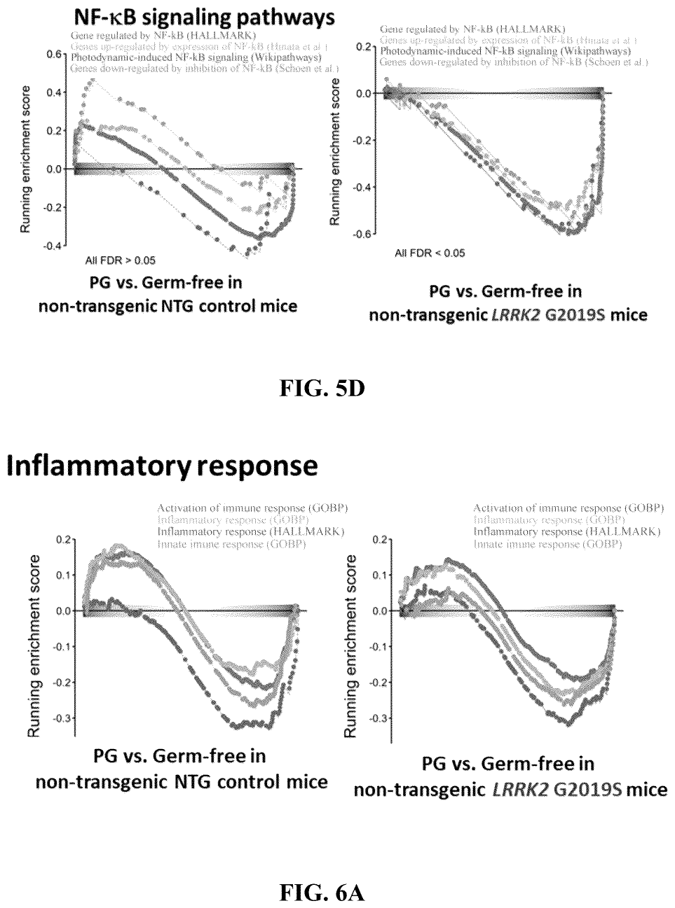

FIGS. 5A-5D show that Parabacteroides goldsteinii strain MTS01 (PG) implantation causes gene cluster changes in brain tissue RNA sequences. (5A) changes of gene clusters in LRRK2 G2019S mice with and without PG inoculation. (5B,5C,5D). Parabacteroides goldsteinii strain MTS01 diminished brain inflammation via toll-like receptor (TLR) and nuclear factor kappa B (NF-κB) pathways. N=4 in each group. LRRK2 means Leucine-rich repeat kinase 2.

Consistently, the comparative bulk RNA-seq of brain tissues derived from individual group of mice showed different gene cluster changes after Parabacteroides goldsteinii strain MTS01 treatment (FIG. 5A). Of note, genes related to inflammatory response were negatively enriched in Parabacteroides goldsteinii strain MTS01-treated LRRK2 G2019S mice compared to germ-free PD mice, which was not observed in NTG littermate controls (FIG. 5B). Genes involved in TLR signaling and NF-kB pathways were down-regulated in Parabacteroides goldsteinii strain MTS01-treated mice (FIGS. 5C, D). In summary, these preliminary results reinforce that Parabacteroides goldsteinii strain MTS01 diminished brain inflammation via TLR and NF-κB pathways in the LRRK2 G2019S PD mouse model.

### Example 4

Parabacteroides goldsteinii Strain MTS01 Did not Affect Gut Inflammation but Activate Mitochondrial Function of LRRK2 G2019S Mice

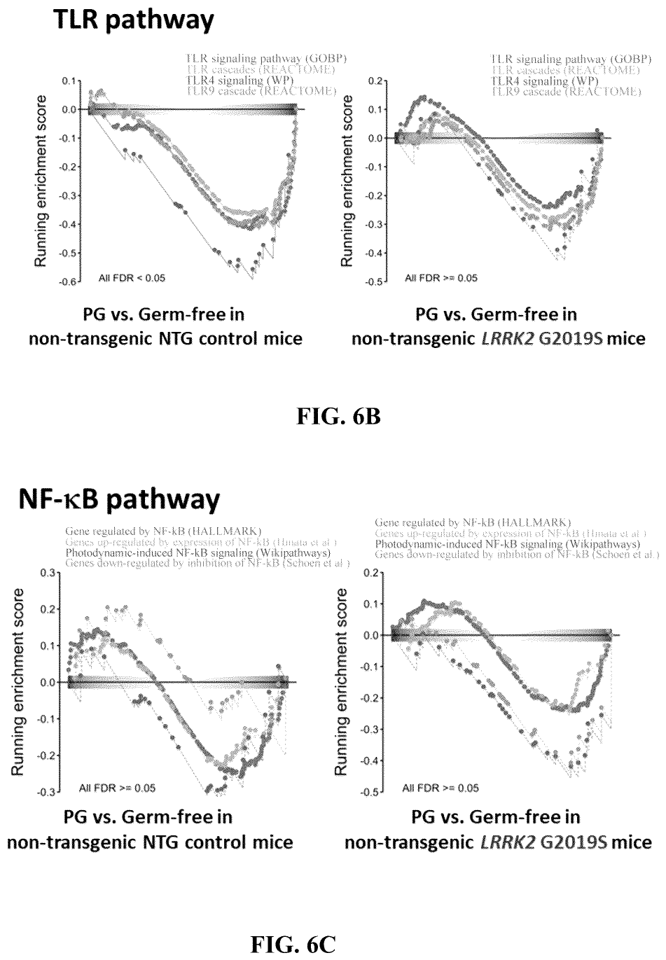

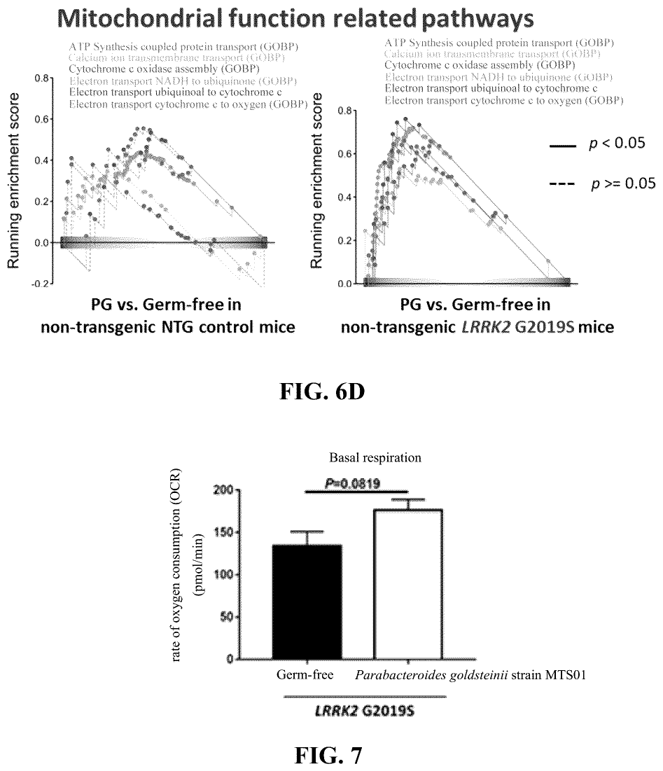

FIGS. 6A-6D show that Parabacteroides goldsteinii strain MTS01 (PG) implantation did not affect gut gene clusters related to inflammatory responses (A), TLR signaling pathways (B) or NF-κB pathways (C) of LRRK2 G2019S mice. Notably, Parabacteroides goldsteinii strain MTS01 activates mitochondrial function in gut tissues of LRRK2 G2019S mice (D). N=4 in each group. LRRK2 means Leucine-rich repeat kinase 2. The four colors represent the four gene-enriched features in the upper right corner of each figure.

Surprisingly, the RNA-seq of gut tissues did not show any differences in gene clusters related to the above-mentioned inflammatory responses, TLR signaling and NF-kB pathways between groups (FIGS. 6A, B, C). Remarkably, gut transcriptome analysis of intestinal tissues in PD mice reveal that Parabacteroides goldsteinii strain MTS01 activate gene clusters related to mitochondrial function, including increasing ATP synthesis, TCA cycle, and mitochondrial organization, and decreasing ROS levels (FIG. 6D).

Transcriptomic analysis of colon tissues of germ-free LRRK2 G2019S mice and those mono-colonized with Parabacteroides goldsteinii strain MTS01 revealed Parabacteroides goldsteinii strain MTS01 significantly increased the expression of gene sets associated with mitochondrial oxidative phosphorylation system (FIGS. 6A-6D). Therefore, we further examined the respiratory function in the isolated mitochondria of murine colon by measuring the rate of oxygen consumption (OCR) using the Seahorse XF24 analyzer, and the activities of germ-free LRRK2 G2019S mice and those mono-colonized with Parabacteroides goldsteinii strain MTS01 were compared. The result is shown in FIG. 7.

FIG. 7 shows the mitochondrial respiratory function of colon tissues of germ-free LRRK2 G2019S mice and those mono-colonized with Parabacteroides goldsteinii strain MTS01. The rate of oxygen consumption (OCR) of isolated mitochondria was determined with the Seahorse XF24 analyzer. The data was analyzed by unpaired t test (n=5 in each group).

The preliminary results revealed that the basal respiration was higher after colonization with Parabacteroides goldsteinii strain MTS01 (FIG. 7), suggesting Parabacteroides goldsteinii strain MTS01 might increase the mitochondrial respiratory activity of colon tissue in PD mice.

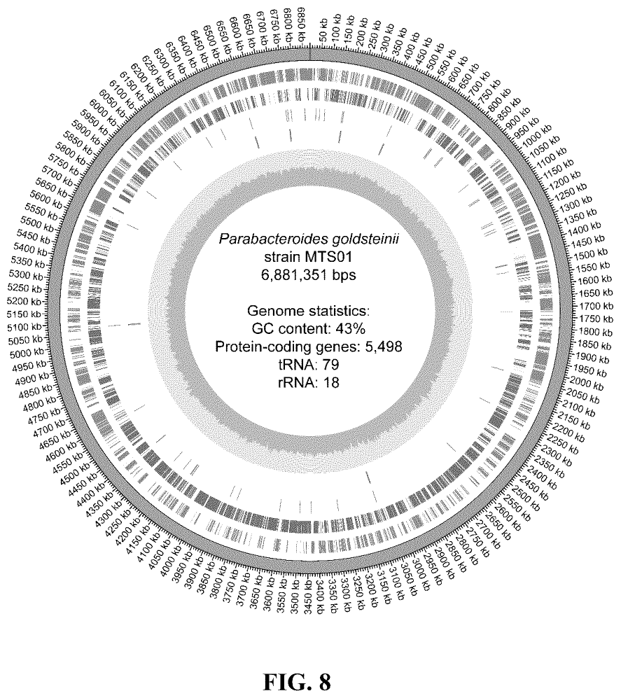

FIG. 8 shows the circular genome map of the whole-genome sequence analysis of the Parabacteroides goldsteinii strain MTS01. The 16S rRNA gene sequence of Parabacteroides goldsteinii strain MTS01 is shown in SEQ ID NO:1.

In summary, the Parabacteroides goldsteinii of the present invention achieves the effect on treating Parkinson's disease through improving locomotor function of the subject in need of treatment for Parkinson's disease, mitigating dopaminergic neuronal degeneration and microglial activation of the subject in need of treatment for Parkinson's disease, alleviating neuroinflammation of the subject in need of treatment for Parkinson's disease, and activating mitochondrial function of the subject in need of treatment for Parkinson's disease.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating Parkinson's disease, comprising administering to a subject in need thereof a pharmaceutical composition comprising an effective amount of Parabacteroides goldsteinii.

2. The method according to claim 1, wherein the Parabacteroides goldsteinii is a live bacterium.

3. The method according to claim 1, wherein the effective amount of Parabacteroides goldsteinii is at least 2×10⁸ CFUs per day for the subject.

4. The method according to claim 1, wherein the Parabacteroides goldsteinii improves locomotor function of the subject in need of treatment for Parkinson's disease.

5. The method according to claim 1, wherein the Parabacteroides goldsteinii mitigates dopaminergic neuronal degeneration and microglial activation of the subject in need of treatment for Parkinson's disease.

6. The method according to claim 1, wherein the Parabacteroides goldsteinii alleviates neuroinflammation of the subject in need of treatment for Parkinson's disease.

7. The method according to claim 1, wherein the Parabacteroides goldsteinii activates mitochondrial function of the subject in need of treatment for Parkinson's disease.

8. The method according to claim 1, wherein the pharmaceutical composition is in a dosage form for oral administration.

9. The method according to claim 1, wherein the pharmaceutical composition is in a dosage form for parenteral administration.

10. The method according to claim 1, wherein the pharmaceutical composition further comprises a pharmaceutically acceptable excipient, carrier, adjuvant and/or food additive.

11. The method according to claim 1, wherein the pharmaceutical composition is taken together with general food, the pharmaceutical composition further comprises bacteria other than the Parabacteroides goldsteinii, and the pharmaceutical composition further comprises an ingredient selected from the group consisting of proteins, monosaccharides, disaccharides, oligosaccharides, polysaccharides, carbohydrates, amino acids, lipids, vitamins, and any combination thereof.

12. The method according to claim 1, wherein the Parabacteroides goldsteinii is a Parabacteroides goldsteinii strain, and the Parabacteroides goldsteinii strain comprises a 16s rRNA gene sequence with at least 95%, 96%, 97%, 98%, 99%, 99.5%, or 99.9% sequence identity to the 16s rRNA gene sequence of Parabacteroides goldsteinii strain MTS01.
